(19) United States
(12) Patent Application Publication
(10) Pub. No.: US 2008/0306162 A1
(43) Pub. Date: Dec. 11, 2008

(54) Treatment of Inflammatory Disorders and Pain

(76) Inventors: Andrew Douglas Baxter, Cambridge (GB); Michael Harvey Lyne, Essex (GB); Robin Mark Bannister, Essex (GB); Elena Lasterra, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/993,910

(22) PCT Filed: Jun. 30, 2006
(86) PCT No.: PCT/GB06/02413

§ 371 Date: Jul. 14, 2008

(30) Foreign Application Priority Data
Jun. 30, 2005 (GB) 0513413.5

(51) Int. Cl. A61K 31/135; C07C 215/68; C07C 215/20; A61P 25/00; A61P 11/00; A61P 29/00; C07D 215/00

(52) U.S. Cl. 514/653; 564/355; 546/152

(57) ABSTRACT

A compound that is useful for the treatment or prevention of a condition such as pain, that is associated with T-cell proliferation or that is mediated by pro- and/or anti-inflammatory cytokines, is of formula (I) wherein R₁ is aryl or heteroaryl optionally substituted with R₅; R₂ is H, alkyl or CH₂OH and can be part of a ring with R₄; R₃ is H, alkyl or CH₂OH and can be part of a ring with R₄; R₄ is H or alkyl or CH₂ (when forming part of a ring with R₂ or R₃); and R₅ is alkyl, CF₃, OH, Oalkyl, OCOalkyl, CONH₂, CN, F, Cl, Br, I, NH₂, NHCHO, NHCONH₂, NHSO₂alkyl, CONH₂, SOMe, CH₂OH or OCONalkyl₂; or a salt thereof.

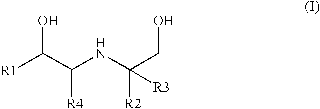

### FIELD OF THE INVENTION

This invention relates to the treatment of inflammatory disorders and pain

### BACKGROUND OF THE INVENTION

Immune driven inflammatory events are a significant cause of many chronic inflammatory diseases where prolonged inflammation causes tissue destruction and results in extensive damage and eventual failure of the effected organ. The cause of these diseases is unknown, so are often called autoimmune, as they appear to originate from an individual's immune system turning on itself. Conditions include those involving multiple organs, such as systemic lupus erythematosus (SLE) and scleroderma. Other types of autoimmune disease can involve specific tissues or organs such as the musculoskeletal tissue (rheumatoid arthritis, ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy, interstitial nephritis), exocrine glands (Sjogrens syndrome and autoimmune pancreatitis) and skin (psoriasis and atopic dermatitis).

In addition, there are chronic inflammatory diseases whose aetiology is more or less known but whose inflammation is also chronic and unremitting. These also exhibit massive tissue/organ destruction and include conditions such as osteoarthritis, These conditions are a major cause of illness in the developing world and poorly treated by current therapies.

Inflammation of skin structures (dermatitis) is a common set of conditions. These diseases are treated using a wide array of therapies, many of which have very severe side-effects.

Current disease modifying treatments (if any), for immune driven conditions, include neutralising antibodies, cytotoxics, corticosteriods, immunosuppressants, antihistamines and antimuscarinics. These treatments are often associated with inconvenient routes of administration and severe side effects leading to compliance issues. Moreover certain drug classes are only effective for certain types of inflammatory diseases; e.g. antihistamines for rhinitis.

### SUMMARY OF INVENTION

Surprisingly, it has been found that compounds of formula (I) are inhibitors of cytokines and possess anti-inflammatory properties as well as work at reducing pain in pain conditions where cytokines are involved. According to the present invention, an inflammatory condition as previously described is treated by the use of such compounds.

According to another aspect of the invention, pain such as acute, chronic or neuropathic pain (including, but not limited to, pain associated with cancer, surgery, arthritis, dental surgery, painful neuropathies, trauma, musculo-skeletal injury or disease, and visceral diseases) and migraine headache in mammals, can be treated by the use of a compound of formula (I)

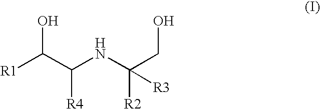

wherein:

R₁ is aryl or heteroaryl optionally substituted with R₅;

R₂ is H, alkyl or CH₂OH and can be part of a ring with R₄;

R₃ is H, alkyl or CH₂OH and can be part of a ring with R₄;

R₄ is H or alkyl or CH₂ (when forming part of a ring with R₂ or R₃); and

R₅ may be alkyl, CF₃, OH, Oalkyl, OCOalkyl, CONH₂, CN, F, Cl, Br, I, NH₂, NO₂, NHCHO, NHCONH₂, NHSO₂alkyl, CONH₂, SOMe, CH₂OH or OCONalkyl₂; or a salt thereof.

### DESCRIPTION OF THE INVENTION

The term “aryl” as used herein refers to optionally substituted aromatic ring systems comprising six to ten ring atoms, and optionally substituted polycyclic ring systems having two or more cyclic rings at least one of which is aromatic. This term includes, for example, phenyl and naphthyl.

The term “heteroaryl” as used herein refers to aromatic ring systems of five to ten atoms at least one atom of which is selected from O, N and S. The term includes, for example, furanyl, thiophenyl, pyridyl, indolyl, quinolyl and the like.

The term “alkyl” as used herein refers to an optionally substituted straight or branched chain alkyl moiety having from one to six carbon atoms. The term includes, for example, methyl, ethyl, propyl, isopropyl, butyl, tert-butyl, pentyl, hexyl and the like. The substituents may be the same or different in each occurrence and selected from halogen and the like. “C₁₋₆ alkyl” has the same meaning.

The “ring” may be aryl, heteroaryl, cycloalkyl or heterocycloalkyl.

The term “cycloalkyl” as used herein refers to a saturated alicyclic moiety having from three to six carbon atoms. The term includes, for example, cyclopropyl, cyclobutyl, cyclopentyl, cyclohexyl and the like. The group may be optionally substituted by any substituent described herein.

The term “heterocycloalkyl” as used herein refers to a saturated heterocyclic moiety having from three to seven carbon atoms and one or more heteroatoms selected from the group N, O, S, P and Si. The term includes, for example, azetidinyl, pyrrolidinyl, tetrahydrofuranyl, piperidinyl and the like. The group may be optionally substituted by any substituent described herein.

Compounds of formula (I) suitable for use in the invention include (but are not limited to) novel compounds such as:
2-[2-(4-hydroxy-3-hydroxymethylphenyl)-2-hydroxyethylamino]-2-methylpropan-1-ol
2-[2-(4-aminophenyl)-2-hydroxyethylamino]-butan-1-ol
2-[2-(3,5-dimethylcarbamoyloxyphenyl)-2-hydroxyethylamino]-2-methylpropan-1-ol
2-[2-(3,4-dihydroxyphenyl)-1-ethyl-2-hydroxyethylamino]-propan-1-ol
2-[2-(5,6,7,8-tetrahydro-2-naphthyl)-2-hydroxyethylamino]-butan-1-ol
2-[2-(2,5-dimethoxyphenyl)-1-methyl-2-hydroxyethylamino]-2-methylpropan-1-ol
2-[2-(4-hydroxy-3-ureylphenyl)-2-hydroxyethylamino]-2-methylpropan-1-ol
2-[2-(4-amino-3-cyanophenyl)-2-hydroxyethylamino]-propan-1-ol
2-[2-(2-chlorophenyl)-2-hydroxyethylamino]-propan-1-ol
2-[2-(3,4-dihydroxyphenyl)-2-hydroxyethylamino]-2-methylpropan-1-ol
2-[2-(4-hydroxyphenyl)-2-hydroxyethylamino]-propan-1-ol
2-[2-(3,4-diacetylphenyl)-2-hydroxyethylamino]-propan-1-ol
2-[2-(3,4-dichlorophenyl)-2-hydroxyethylamino]-propan-1-ol
2-[2-(2,5-dimethoxyphenyl)-1-methyl-2-hydroxyethylamino]-propan-1-ol
2-[2-(3,4-dihydroxyphenyl)-1-ethyl-2-hydroxyethylamino]-propan-1-ol
2-[2-(4-hydroxy-3-methoxyphenyl)-2-hydroxyethylamino]-propan-1-ol
2-[2-(3-hydroxyphenyl)-2-hydroxyethylamino]-propan-1-ol
2-[2-(4-nitrophenyl)-2-hydroxyethylamino]-propan-1-ol
2-[2-(8-hydroxyquinolin-5-yl)-2-hydroxyethylamino]-propan-1-ol
2-[2-(8-hydroxy-1H-quinoline-2-one-5-yl)-2-hydroxyethylamino]-propan-1-ol
2-[2-(4-hydroxy-3-methanesulphonamidephenyl)-2-hydroxyethylamino]-propan-1-ol
2-[2-(4-methanesulphonamidephenyl)-2-hydroxyethylamino]-propan-1-ol
2-[2-(2-chloro-4-hydroxyphenyl)-2-hydroxyethylamino]-2-methylpropan-1-ol
2-[2-(2-fluorophenyl)-2-hydroxyethylamino]-2-methylpropan-1-ol
2-[2-(4-hydroxy-3-methylsulfonylphenyl)-2-hydroxyethylamino]-2-methylpropan-1-ol
2-[2-(4-amino-3,5-dicholorophenyl)-2-hydroxyethylamino]-2-methylpropane-1,3-diol and
2-[2-(4-amino-3,5-dicholorophenyl)-2-hydroxyethylamino]-2-(hydroxymethyl)propane1,3-diol.

Compounds of formula (I) suitable for use in the invention that are not novel (in Registry) but have not previously been proposed for use in therapy include:
2-[2-(3,5-ditertbutylcarbonyloxyphenyl)-2-hydroxyethylamino]-2-methylpropan-1-ol
2-[2-(3,5-diisopropylcarbonyloxyphenyl)-2-hydroxyethylamino]-2-methylpropan-1-ol
2-[2-(3,5-dihydroxyphenyl)-2-hydroxyethylamino]-propan-1-ol and
2-[2-(3,5-dihydroxyphenyl)-2-hydroxyethylamino]-2-methylpropan-1-ol.

Other compounds of formula (I) suitable for use in the invention include:
2-[2-(4-amino-3,5-dicholorophenyl)-2-hydroxyethylamino]-2-methylpropan-1-ol
   (although the single isomers of this compound are novel)
2-[2-(3-cholorophenyl)-2-hydroxyethylamino]-2-methylpropan-1-ol
2-[2-phenyl-2-hydroxyethylamino]-2-methylpropan-1-ol (Fepradinol)
2-[2-(2-chlorophenyl)-2-hydroxyethylamino]-2-methylpropan-1-ol
2-[2-(3-chloro-4-amino-5-trifluoromethylphenyl)-2-hydroxyethylamino]-2-methylpropan-1-ol
2-[2-(naphthalene-2-yl)-2-hydroxyethylamino]-propan-1-ol and
2-[2-(3,4-dihydroxyphenyl)-2-hydroxyethylamino]-propan-1-ol

It is understood that the invention refers to salts, e.g. the hydrochloride, metabolites and pro-drugs thereof, as well as any diastereomers and enantiomers of (I).

Some of the compounds of formula (I) have antihypertensive, vasodilator, sympathomimetic, bronchodilator or cardiostimulant activity through agonism and antagonism at alpha and beta adrenoceptors. These agents have at least one chiral centre and their activity at the alpha or beta adrenoceptors resides mainly or solely in one of the enantiomers. If the molecule has more than one chiral centre, the activity at the alpha or beta adrenoceptors resides mainly in one of the diastereomers.

According to another aspect of the invention, a preferred diastereomer or enantiomer of (I) has little or no activity at the α or β adrenoceptors. This activity may be determined by use of the appropriate in vitro assay.

The compounds of formula (I) according to the invention are used to treat inflammatory diseases including, but not exclusive to, autoimmune diseases involving multiple organs, such as systemic lupus erythematosus (SLE) and scleroderma, specific tissues or organs such as the musculoskeletal tissue (rheumatoid arthritis, ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin dependent diabetes mellitus), the adrenal gland (Addison's disease), the kidney (Goodpasture's syndrome, IgA nephropathy, interstitial nephritis), exocrine glands (Sjogrens syndrome and autoimmune pancreatitis) and skin (psoriasis and atopic dermatitis), chronic inflammatory diseases such as osteoarthritis, periodontal disease, diabetic nephropathy, chronic obstructive pulmonary disease, artherosclerosis, graft versus host disease, chronic pelvic inflammatory disease, endometriosis, chronic hepatitis and tuberculosis, IgE-mediated (Type I) hypersensitivities such as rhinitis, asthma, anaphylaxis and dermatitis. Dermatitis conditions include actinic keratosis, acne rosacea, acne vulgaris, allergic contact dermatitis, angioedema, atopic dermatitis, bullous pemiphigoid, cutaneous drug reactions, erythema multiforme, lupus erythrametosus, photodermatitis, psoriasis, psoriatic arthritis, scleroderma and urticaria.

These compounds may be used according to the invention when the patient is also administered or in combination with another therapeutic agent selected from corticosteroids (examples including cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone), disease modifying anti-rheumatic drugs (DMARDs) (examples including azulfidine, aurothiomalate, bucillamine, chlorambucil, cyclophosphamide, leflunomide, methotrexate, mizoribine, penicillamine and sulphasalazine), immunosuppressants (examples including azathioprine, cyclosporine and mycophenolate), COX inhibitors (examples including aceclofenac, acemetacin, alcofenac, alminoprofen, aloxipirin, amfenac, aminophenazone, antraphenine, aspirin, azapropazone, benorilate, benoxaprofen, benzydamine, butibufen, celecoxib, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, mefenamic acid, metamizole, mofebutazone, mofezolac, nabumetone, naproxen, nifenazone, oxametacin, phenacetin, pipebuzone, pranoprofen, propyphenazone, proquazone, rofecoxib, salicylamide, salsalate, sulindac, suprofen, tiaramide, tinoridine, tolfenamic acid and zomepirac), neutralising antibodies (examples including etanercept and infliximab), and antibiotics (examples including doxycycline and minocycline).

Compounds of formula (I) exhibit analgesic activity in animal models. The activity of these compounds may be determined by the use of the appropriate in vivo assay.

This invention also relates to a method of treatment for patients (including man and/or mammalian animals raised in the dairy, meat or fur industries or as pets) suffering from chronic, acute or neuropathic pain; and more specifically, a method of treatment involving the administration of the analgesic of formula (I) as the active constituent.

Accordingly, the compounds of formula (I) can be used among other things in the treatment of pain conditions such as acute and chronic pain (as well as, but not limited to, pain associated with cancer, surgery, arthritis, dental surgery, trauma, musculo-skeletal injury or disease, visceral diseases) and migraine headache. Additionally the painful conditions can be neuropathic (post-herpetic neuralgia, diabetic neuropathy, drug induced neuropathy, HIV mediated neuropathy, sympathetic reflex dystrophy or causalgia, fibromyalgia, myofacial pain, entrapment neuropathy, phantom limb pain, trigeminal neuralgia. Neuropathic conditions include central pain related to stroke, multiple sclerosis, spinal cord injury, arachnoiditis, neoplasms, syringomyelia, Parkinson's and epilepsia.

It will often be advantageous to use compounds of formula (I) in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

Any suitable route of administration can be used. For example, any of oral, topical, parenteral, ocular, rectal, vaginal, inhalation, buccal, sublingual and intranasal delivery routes may be suitable. The dose of the active agent will depend on the nature and degree of the condition, the age and condition of the patient and other factors known to those skilled in the art. A typical dose is 10-100 mg given one to three times per day.

Compounds of formula (I) may be prepared by either of the schematic routes illustrated:

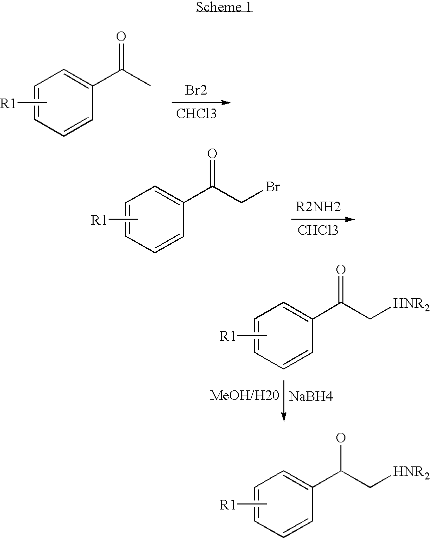

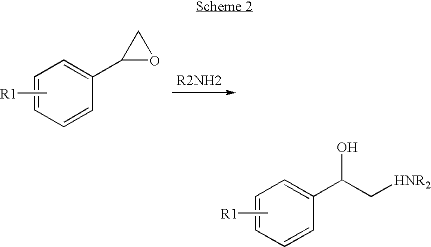

It will be appreciated that other compounds of formula (I) can be made analogously, using compounds and procedures that are known or can be made by those of ordinary skill in the art.

The following Examples illustrate the invention.

### EXAMPLE 1

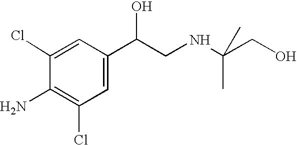

### 4-amino-bromo-3,5-dichloroacetophenone

Bromine (63 ml, 1.22 mol) was added to a mixture of 4-amino-3,5-dichloroacetophenone (250 g, 1.22 mol) in CHCl₃ (3 L) at room temperature. The mixture was stirred for 1 h then EtOH (500 ml) was added. The mixture was cooled to 0° C. and stirred for 1 h. The precipitate was filtered and air-dried (4.7 g, 67%). ¹H NMR (400 MHz, DMSO): 4.77 (2H, s), 6.61 (2H, bs), 7.86 (2H, s); ¹³C NMR (100 MHz, DMSO): 63.39, 117.89, 128.57, 129.75, 146.17, 195.99.

### 1-(4-amino-3,5-dichlorophenyl)-2-(1-hydroxy-2-methylpropan-2-ylamino)ethanone

2-amino-2-methyl-propan-1-ol (180 ml, 2.49 mol) was added to a mixture of the previous product (237 g, 0.83 mol) in chloroform (650 ml). The mixture was stirred at room temperature for 2 h, then water (380 ml) was added. The mixture was stirred for 1 h, and then the solid was filtered. The solid was triturated with water (1 L) to give the desired compound (223 g, 91%). ¹H NMR (400 MHz, DMSO): 0.94 (6H, s), 3.18 (2H, d J=4.4 Hz), 3.93 (2H, s), 4.55 (1H, m), 6.40 (2H, s), 7.84 (2H, s); ¹³C NMR (100 MHz, DMSO): 24.21, 48.87, 53.73, 68.52, 117.92, 124.57, 125.79, 128.62, 146.07, 195.30; LC-MS: 291, 292, 293 (M+H⁺).

### 2-[2-(4-amino-3,5-dichlorophenyl)-2-hydroxyethylamino]-2-methylpropan-1-ol

A slurry of sodium borohydride (35.5 g, 0.93 mol) in water (80 ml) was added slowly to a mixture of the previous product (121 g, 0.41 mol) in MeOH (775 ml) and water (390 ml) at 0° C. The mixture was stirred for 2 h at room temperature and evaporated to dryness. The residue was slurried in EtOH (1.5 L), filtered and the filtrate was evaporated to dryness to give a white solid (76 g, 62%). ¹H NMR (400 MHz, CDCl₃): 1.03 (6H, s), 2.53-2.58 (1H, m), 2.71-2.76 (1H, m), 4.39 (2H, bs), 4.46-4.49 (1H, m), 7.17 (2H, m); ¹³C NMR (100 MHz, CDCl₃): 24.03, 24.19, 49.64, 53.66, 68.95, 72.01, 119.64, 125.47, 133.04, 139.45; LC-MS: 293 (M+H⁺).

### Single enantiomers of 2-[2-(4-amino-3,5-dichlorophenyl)-2-hydroxyethylamino]-2-methylpropan-1-ol (1)

2-(2-(4-amino-3,5-dichlorophenyl)-2-hydroxyethylamino)-2-methylpropan-1-ol (2.0 g) was purified on a CHIRALPAK AS-H 5 μm (250 mm×20 mm) chromatography column, with an eluent of 90/10 CO2/(MeOH+1% diethylamine), a flow rate of 60 ml/min and UV wavelength detection at 230 nm, at a temperature of 25° C. and an outlet pressure of 150 bars. 0.95 g of a yellow oil was isolated as the first eluting peak, HPLC 95.8%, enantiomeric excess >99.0. 0.95 g of a yellow oil was isolated as the second eluting peak, HPLC 92.3%, enantiomeric excess >99.0.

### EXAMPLE 2

### 2-(2-phenyl-2-hydroxyethylamino)-2-methyl-propan-1-ol

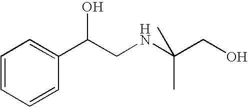

To a 250 ml 3-necked flask equipped with magnetic stirrer, reflux condenser, pressure equalizing dropping funnel and under nitrogen was charged 2-amino-2-methyl-1-propanol (10.0 ml, 0.112 mol). Styrene oxide (7.21 ml, 0.05 mol) was added dropwise and. the solution was heated overnight, (external oil bath temperature 105° C.). TLC analysis 95:5 (DCM:MeOH) indicated no starting material present.

After cooling to room temperature, the precipitated solid was dissolved in boiling ethanol. Petroleum ether (few mls) was added to the stirring solution and a white solid precipitated out. This was collected by filtration, and the crude product was purified by recrystallisation in boiling water. A white solid was afforded (5.30 g, 23.0%). NMR and LCMS analysis were consistent with the desired product. Further crops of the pure material could be obtained from the aqueous filtrate if necessary.

¹H NMR (500 MHz, MeOD) δ=1.07 (s, 6H, 2×CH₃), 2.69-2.77 (m, 2H, NCH₂), 3.32-3.35 (d, 1H, J=11 Hz CH₂O), 3.41-3.43 (d, 1H, J=11 Hz, CH₂O), 4.71-4.74 (dd, 1H, J=4.2 Hz, J=8.7 Hz, CH), 7.26-7.41 (m 5H, Ar).

¹³C NMR spectrum (125 MHz, MeOD) δ=23.22, 24.07, 50.69, 54.69, 69.28, 74.40, 127.07, 128.61, 129.42, 144.71.

### EXAMPLE 3

### 2-[2-(4-fluorophenyl)-2-hydroxyethylamino]-2-methylpropan-1-ol

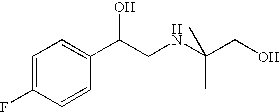

To a 250 mL 3-necked flask equipped with magnetic stirrer, reflux condenser, pressure equalizing dropping funnel and under nitrogen was charged 2-amino-2-methyl-1-propanol (5.00 g, 0.11 mol). 2-(4-Fluorophenyl)oxirane (3.31 mL, 0.05 mol) was added dropwise and the solution was heated overnight (external oil bath temperature=105° C.). TLC analysis (95:5) (DCM:MeOH) indicated no starting material present.

After cooling to room temperature, the precipitated solid was collected by filtration and purified by recrystallisation from boiling water. A white solid was afforded (2.33 g, 21%). NMR and LCMS analyses were consistent with the desired product. Further crops of the pure material could be obtained from the aqueous filtrate if necessary.

¹H NMR (500 MHz, MeOD) δ=1.07 (s, 6H, 2×CH₃), 2.68-2.75 (m, 2H, NCH₂), 3.33-3.36 (d, 1H, J=11.0 Hz CH₂O), 3.41-3.43 (d, 1H, J=10.9 Hz, CH₂O), 4.70-4.73 (dd, 1H, J=4.3 Hz, J=8.7 Hz, CH), 7.07-7.10 (m 2H, Ar), 7.40-7.43 (m, 2H, Ar).

The following studies provide evidence on which the present invention is based.

### Beta2 Agonism Functional Assay

Guinea-pig trachea ring preparations were suspended in Kreb's solution containing indomethacin. After 15 minutes stabilisation, the preparations were repeated contracted using carbachol and simultaneously treated with increasing cumulative doses test compounds (0.1 nM to 0.1 μM). Beta2 agonism for each test compound was determined by its dose-dependant inhibition of carbachol-stimulated tracheal muscle twitch.

The two enantiomers of 2-[2-(4-amino-3,5-dichlorophenyl)-2-hydroxyethylamino)-2-methyl]-propan-1-ol when tested in the beta agonism functional assay gave IC₅₀'s of 2.1 μM and 11.9 nM. Formeterol has an IC₅₀ of 2.2 nM in this system.

### LPS Mouse Assay

7 week old Balb C ByJ mice (24-28 g) were administered, either by i.p. (5 ml/kg) or oral (10 ml/kg) administration, with vehicle or test article. 30 minutes later these animals were challenged with an intraperitoneal injection of 1 mg/kg LPS. 2 hours after LPS challenge blood samples were collected under light isoflurane anaesthesia into normal tubes by retro-orbital puncture. Samples were allowed to clot at room temperature and then spun at 6000 g for 3 min at 4° C. Serum was stored at −20° C. until use. Serum TNFα and IL-10 levels were analysed in duplicate by ELISA technique.

The two enantiomers of 2-[2-(4-amino-3,5-dichlorophenyl)-2-hydroxyethylamino]-2-methylpropan-1-ol gave the following results in the LPS mouse assay; 65% and 13% inhibition of TNFα production at 10 mg/kg, and 78 and 18% inhibition of TNFα production at 30 mg/kg. IL-10 production was potentiated by 420% and 137% at 10 mg/kg and 336 and 132 at 30 mg/kg. The racemate inhibited TNFα production by 60% and 61% at 10 and 30 mg/kg respectively and potentiated IL-10 production by 417% and 392% at 10 and 30 mg/kg respectively.

2-[2-Phenyl-2-hydroxyethylamino]-2-methylpropan-1-ol inhibited TNFα production by 47% and 58% at 10 and 30 mg/kg respectively, and potentiated IL-10 production by 345% and 366% at the same doses.

2-[2-(4-Fluorophenyl)-2-hydroxyethylamino]-2-methylpropan-1-ol inhibited TNFα production by 37% and 37% at 10 and 30 mg/kg respectively, and potentiated IL-10 production by 106% and 125% at the same doses.

### Carrageenan Paw Assay

Fasted (18 hour) male Wistar rats (105-130 g) were weighed and a basal mercury plethysmometer reading was taken of the right hind paw by submerging the paw in the mercury up to the tibiotarsal joint. Subsequently, vehicles, reference items and test articles were administered by oral gavage (10 ml/kg). Half an hour after treatment, 0.1 ml of 2% carrageenan in 0.9% saline was injected into the subplanatar area of the right hind paw. The right paw was measured again with the plethysmometer at 1, 2, 3, 4 and 5 hours after carrageenan administration.

One of the enantiomers of 2-[2-(4-amino-3,5-dichlorophenyl)-2-hydroxyethylamino]-2-methylpropan-1-ol showed a clear anti-inflammatory dose response curve in the carageenan paw assay. The inhibition was 18%, 28%, 26%, 59% and 71% at 0.3, 1, 3, 10 and 30 mg/kg, respectively. Ibuprofen in the same assay showed 38% anti-inflammatory at 100 mg/kg.

1. A method for the treatment or prevention of a condition associated with T-cell proliferation or that is mediated by pro- and/or anti-inflammatory cytokines, wherein said method comprises administering, to a patient in need of such treatment or prevention, a compound of formula (I) 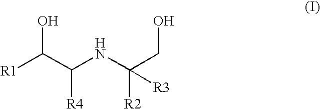
wherein
R₁ may be aryl or heteroaryl optionally substituted with R₅;
R₂ may be H, alkyl or CH₂OH and can be part of a ring with R₄;
R₃ may be H, alkyl or CH₂OH and can be part of a ring with R₄;
R₄ may be H or alkyl or CH₂ (when forming part of a ring with R₂ or R₃); and
R₅ may be alkyl, CF₃, OH, Oalkyl, OCOalkyl, CONH₂, CN, halogen, NH₂, NO₂, NHCHO, NHCONH₂, NHSO₂Me, CONH₂, or SOMe;
or a salt thereof.

2. The method according to claim 1, wherein the condition is a chronic degenerative disease.

3. The method according to claim 1, wherein the condition is a chronic demyelinating disease.

4. The method according to claim 1, wherein the condition is a respiratory disease.

5. The method according to claim 1, wherein the condition is an inflammatory bowel disease (IBD).

6. The method according to claim 1, wherein the condition is a dermatological condition.

7. The method according to claim 1, wherein the condition is a dental disease.

8. The method according to claim 1, wherein the condition is diabetic nephropathy, lupus nephritis, IgA nephropathy or glomerulonephritis.

9. The method according to claim 1, wherein the condition is systemic lupus erythematosus (SLE).

10. The method according to claim 1, wherein the condition is graft vs host disease.

11. The method according to claim 1, wherein the condition is a pain condition.

12. The method according to claim 11, wherein the pain condition is chronic pain such as chronic back pain, malignant pain, chronic headache or arthritic pain.

13. The method according to claim 11, wherein the pain condition is acute pain, post-traumatic pain or acute disease-induced pain.

14. The method according to claim 11, wherein the pain condition is neuropathic pain.

15. The method according to claim 1, wherein the compound is chiral and is in the form of the enantiomer or diastereomer that has relatively little or no activity at the α or β adrenoceptor.

16. The method according to claim 1, wherein the patient is also administered another therapeutic agent selected from corticosteroids, cytotoxics, antibiotics, immunosuppressants, non-steroidal anti-inflammatory drugs, narcotic analgesics, local anaesthetics, NMDA antagonists, neuroleptics, anti-convulsants, anti-spasmodics, anti-depressants and muscle relaxants.

17. The method according to claim 16, wherein the compound and said another agent are provided in combination.

18. A compound selected from the group consisting of:
2-[2-(4-hydroxy-3-hydroxymethylphenyl)-2-hydroxyethylamino]-2-methylpropan-1-ol;
2-[2-(4-aminophenyl)-2-hydroxyethylamino]-butan-1-ol;
2-[2-(3,5-dimethylcarbamoyloxyphenyl)-2-hydroxyethylamino]-2-methylpropan-1-ol;
2-[2-(3,4-dihydroxyphenyl)-1-ethyl-2-hydroxyethylamino]-propan-1-ol;
2-[2-(5,6,7,8-tetrahydro-2-naphthyl)-2-hydroxyethylamino]-butan-1-ol;
2-[2-(2,5-dimethoxyphenyl)-1-methyl-2-hydroxyethylamino]-2-methylpropan-1-ol;
2-[2-(4-hydroxy-3-ureylphenyl)-2-hydroxyethylamino]-2-methylpropan-1-ol;
2-[2-(4-amino-3-cyanophenyl)-2-hydroxyethylamino]-propan-1-ol;
2-[2-(2-chlorophenyl)-2-hydroxyethylamino]-propan-1-ol;
2-[2-(3,4-dihydroxyphenyl)-2-hydroxyethylamino]-2-methylpropan-1-ol;
2-[2-(4-hydroxyphenyl)-2-hydroxyethylamino]-propan-1-ol;
2-[2-(3,4-diacetylphenyl)-2-hydroxyethylamino]-propan-1-ol;
2-[2-(3,4-dichlorophenyl)-2-hydroxyethylamino]-propan-1-ol;
2-[2-(2,5-dimethoxyphenyl)-1-methyl-2-hydroxyethylamino]-propan-1-ol;
2-[2-(3,4-dihydroxyphenyl)-1-ethyl-2-hydroxyethylamino]-propan-1-ol;
2-[2-(4-hydroxy-3-methoxyphenyl)-2-hydroxyethylamino]-propan-1-ol;
2-[2-(3-hydroxyphenyl)-2-hydroxyethylamino]-propan-1-ol;
2-[2-(4-nitrophenyl)-2-hydroxyethylamino]-propan-1-ol;
2-[2-(8-hydroxyquinolin-5-yl)-2-hydroxyethylamino]-propan-1-ol;
2-[2-(8-hydroxy-1H-quinoline-2-one-5-yl)-2-hydroxyethylamino]-propan-1-ol;
2-[2-(4-hydroxy-3-methanesulphonamidephenyl)-2-hydroxyethylamino]-propan-1-ol;
2-[2-(4-methanesulphonamidephenyl)-2-hydroxyethylamino]-propan-1-ol;
2-[2-(2-chloro-4-hydroxyphenyl)-2-hydroxyethylamino]-2-methylpropan-1-ol;
2-[2-(2-fluorophenyl)-2-hydroxyethylamino]-2-methylpropan-1-ol;
2-[2-(4-hydroxy-3-methylsulfonylphenyl)-2-hydroxyethylamino]-2-methylpropan-1-ol;
2-[2-(4-amino-3,5-dicholorophenyl)-2-hydroxyethylamino]-2-methylpropane 1,3-diol; and
2-[2-(4-amino-3,5-dicholorophenyl)-2-hydroxyethylamino]-2-(hydroxymethyl)Propane1,3-diol.

19. The method, according to claim 2, wherein said chronic degenerative disease is rheumatoid arthritis, osteoarthritis or osteoporosis.

20. The method, according to claim 3, wherein the chronic demyelinating disease is multiple sclerosis.

21. The method, according to claim 4, wherein the respiratory disease is asthma or chronic obstructive pulmonary disease.

22. The method, according to claim 5, wherein the IBD is colitis or Crohn's disease.

23. The method, according to claim 6, wherein the dermatological condition is psoriasis, scleroderma or atopic dermatitis.

24. The method, according to claim 7, wherein said dental disease is periodontal disease or gingivitis.

25. The method, according to claim 12, wherein said chronic pain is migraine or cluster headaches.

26. The method, according to claim 13, wherein said acute pain is post operative pain, post-traumatic pain or acute disease induced pain.

27. The method, according to claim 1, wherein R₁ is phenyl optionally substituted with one or more of F, Cl and NH.

28. The method, according to claim 27, wherein the compound is fepradinol.
